(19) United States
(12) Patent Application Publication
(10) Pub. No.: US 2013/0209537 A1
(43) Pub. Date: Aug. 15, 2013

(54) CONTROLLED-RELEASE ANTIBIOTIC NANOPARTICLES FOR IMPLANTS AND BONE GRAFTS

(71) Applicant: Patty Fu-Giles, University Park, IL (US)

(72) Inventor: Patty Fu-Giles, University Park, IL (US)

(21) Appl. No.: 13/574,033

(22) PCT Filed: Jul. 1, 2011
(86) PCT No.: PCT/US2011/042776

§ 371 Date: Apr. 25, 2013

Related U.S. Application Data
(60) Provisional application No. 61/360,802, filed on Jul. 1, 2010.

(51) Int. Cl. A61L 27/54

(52) U.S. Cl. CPC A61L 27/54
USPC 424/423; 514/2.9; 514/263.38; 514/628

(57) ABSTRACT

The present invention relates to the preparation and use of antibiotic-containing nanoparticles for coating an implant including cranial implants and bone graft sites to provide for the extended release of antibiotics to treat infection.

### CROSS-REFERENCE TO RELATED APPLICATION

This application claims priority from U.S. Provisional Application Ser. No. 61/360,802, filed Jul. 1, 2010.

### TECHNICAL FIELD

The present invention relates to a nanoparticulate delivery system for the controlled release of antibiotics from implants and, in particular, from cranial implant and bone graft sites.

### BACKGROUND OF THE INVENTION

Polymethylmethacrylate (PMMA) has been used in orthopedic surgery for decades as a cement for securing prosthetic implants and more recently as a delivery agent for local high-dose antibiotics to treat soft tissue and bone infections. Antibiotics are eluted from the surface and pores of the cement and through microcracks in the cement. However, because PMMA is non-bioabsorbable, a significant portion of the antibiotic dose contained within the cement is often not available to effectively treat infections. As a result, surgical use of PMMA for antibiotic delivery sometimes requires multiple replacement of PMMA in the form of antibiotic-loaded beads.

It has been shown that drug delivery systems using nanoparticle-encapsulated antibiotics can improve antimicrobial efficacy against drug-resistant strains. (Torchilin, 2001; Nandi et al., 2003 Garay-Jimenez et al., 2009). Nanoparticles such as liposomes and micelles have been used to protect drugs within a relatively impermeable bilayer or multilayer environment and to prolong release times by isolating the encapsulated drugs from systematic degrading enzymes. Liposomes, micelles and other nanoparticles can be taken up by cells without overt cytotoxic effects, thus enhancing the cellular uptake of the encapsulated material and promoting diffusion across the bacterial or viral envelope. (Torchilin, 2001; Muller-Goymann, 2004; Wang, 2009). Moreover, such nanoparticles are natural, biodegradable and non-toxic. However, this type of nanoparticulate system has not been extended to use in bone replacement. In particular, there is a need for an effective delivery system for antibiotics for preventing and treating infections related to craniofacial and traumatic brain injuries.

The major focus of nanoparticulate drug delivery systems to date has related to nanoparticles as polymeric carriers for anticancer agents or for gene delivery and tissue engineering. (Henry, 2002; Richter, 2010). There is an advantage to providing antibiotics in the form of nanoparticles to provide for prolonged release in treating infection. Thus, there is a need for a system including antibiotics encapsulated within liposomes, micelles and other nanoparticles to treat and alleviate post-surgical and post-transplantation infections. This would avoid the need for multiple replacement of antibiotic-loaded beads which is impractical and undesirable with cranial implants.

### SUMMARY OF THE INVENTION

The present invention relates to a nanoparticulate system for delivering antibiotics in a locally applied and extended-release manner to patients receiving bone implants and, in particular, cranial replacement implants and bone grafts. The method of the present invention includes: (1) encapsulating a hydrophobic antibiotic (for example, rifampicin and chloramphenicol) and/or a hydrophilic antibiotic (for example, vancomycin and acyclovir) into antibiotic-containing nanoparticles; (2) incorporating the antibiotic-containing nanoparticles into a polymeric coating material (for example, nitrocellulose plus 7.0% (w/v) polyvinylpyrrolidone) with a volatile carrier solvent (ethyl acetate or ethanol); and (3) applying the product of step (2) to an implant before surgery. In a preferred embodiment, the implant comprises a polymethylmethacrylate (PMMA) cranial implant or a hydroxyapatite (HA) bone grafting material.

Other antibiotics including novobiocin, spectinomycin, trimethoprim, erythromycin, doxycycline, minocycline, amphotericin B, gentamicin, gentamicin sulfate, tobramycin, ampicillin, penicillin, ethambutol, clindamycin, and cephalosporins including cefazolin, ceftriaxone and cefotaxime can also be used, including pharmacologically acceptable salts and acids thereof.

When the volatile carrier solvent evaporates, the polymeric coating material with embedded antibiotic nanoparticles forms a thin film that attaches to the surface of the implant or grafting material. Local application of encapsulated antibiotics directly to an implant or surgical site provides a non-oral, non-intravenous, controlled time-release method for providing continuous administration of an antibiotic over a prescribed time period. The invention provides a novel chemotherapeutic approach in more efficient, effective doses for the prevention and treatment of bacterial, fungal and viral infections that often occur in implants, particularly in cranial/bone transplant patients.

An advantage of the present invention is the development of a novel nanovesicular drug delivery system that offers improved pharmaceutical properties, is easily integrated onto the surface of PMMA and bone grafting implants prior to surgery, and facilitates the delivery of antibiotics to prevent post-operative infections.

This specific targeting drug delivery system helps reduce dangerous side effects. It also eliminates the time that otherwise is needed for the drugs to be processed by the liver. Therefore, a reduced amount of the drug will produce comparable beneficiary effects compared to intravenous or oral administration of the drug.

Furthermore, the present delivery system can be customized based on the needs of the patient by varying the entrapped antibiotics and the mixture of nanostructures in the drug delivery assay. Finally, all nanovesicles in this system are composed of organic materials, which are already used in many FDA-approved drug delivery systems.

### BRIEF DESCRIPTION OF THE DRAWINGS

FIG. 1 shows a partial cutaway view of a liposome having a double membrane which can encapsulate both hydrophilic molecules in its core and hydrophobic molecules in its lipid bilayer in aqueous solution;

FIG. 2 shows a partial cutaway view of a micelle including the hydrophobic core and the hydrophilic outer surface or shell which allows the encapsulation of hydrophobic molecules in an aqueous solution;

FIG. 3 is a transmission electron microscopy (TEM) image of encapsulated rifampicin nanoparticles, the lower image showing no aggregation of the nanoparticles within the matrix;

FIG. 4 shows a fluorescence spectrum before (B-D) encapsulation at various pH values and after (A) encapsulation;

FIGS. 5a and 5b, respectively, show the high surface area of commonly used implant materials—polymethylmethacrylate (PMMA) and hydroxyapatite (HA);

FIG. 6 shows the results of a cell penetration study in which human dermal fibroblast cells are incubated with nanoparticles for about 2 hours and then lysed to demonstrate fluorescent readings before (a) and after (b) cell lysis;

FIG. 7 shows transmission electron microscopy (TEM) images of unilamellar liposomes containing Example 1B; and

FIG. 8 shows the relative intensity of fluorescent dye in aqueous solution (A) and encapsulated within reverse micelles (2) formed according to Example 3 and liposomes (3) formed according to Example 2.

### DETAILED DESCRIPTION OF THE INVENTION

FIG. 1 shows a liposome with a double membrane that can encapsulate both hydrophilic molecules in its core and hydrophobic molecules in the lipid bilayer in an aqueous solution. Liposomes are closed lipid bilayer membranes containing an entrapped aqueous volume. Liposomes can comprise unilamellar vesicles with a single membrane bilayer or multilamellar vesicles including onion-like structures with multiple membrane bilayers, each separated from the next by an aqueous layer. The bilayer comprises two lipid monolayers including a hydrophobic tail region and a hydrophilic head region. The structure of the membrane bilayer is such that the hydrophobic (nonpolar) tails of the lipid monolayers orient towards the center of the bilayer while the hydrophilic heads orient towards the aqueous phase.

The original liposome preparation of Bangham et al. (J. Mol. Biol., 1965, 13:238-252) involves suspending phospholipids in an organic solvent which is then evaporated to dryness leaving a phospholipid film on the reaction vessel. An appropriate amount of aqueous phase is then added, the mixture is allowed to “swell,” and the resulting liposomes which comprise multilamellar vesicles (MLVs) are dispersed by mechanical means. This technique provides the basis for the development of the small sonicated unilamellar vesicles described by Papahadjopoulos et al. (Biochem. Biophys. Acta., 1967, 135:624-638), and large unilamellar vesicles.

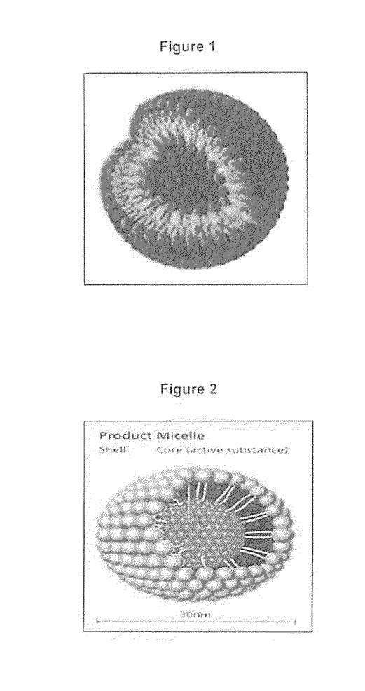

As shown in FIG. 2, a typical micelle has a hydrophobic core and a hydrophilic outer surface or shell allowing the encapsulation of hydrophobic molecules in an aqueous solution. A typical micelle in aqueous solution forms an aggregate with the hydrophilic head regions in contact with the surrounding solvent, entrapping the hydrophobic tail regions in the micelle center. The difficulty of filling all the volume of the interior of the bilayer, while accommodating the area per head group forced on the molecule by the hydration of the lipid head group leads to formation of the micelle. This type of micelle is known as a normal phase micelle (oil-in-water micelle).

Inverse micelles, on the other hand, include hydrophilic head regions positioned at the center of the micelle with the tails extending outwardly (water-in-oil micelle). Inverse (or reverse) micelles, with a hydrophilic core, are created using the microemulsion method. This type of micelle is specifically used to encapsulate hydrophilic materials. In a non-polar solvent, the exposure of the hydrophilic head groups to the surrounding solvent gives rise to a water-in-oil system. As a result, the hydrophilic groups are entrapped in the micelle core and the hydrophobic groups extend away from the center. Inverse micelles are generally smaller, tighter and more stable than regular micelles and liposomes.

A review of methods for producing liposome, micelles and inverse micelles is provided in Liposomes, Marc Ostro, ed., Marcel Dekker, Inc. New York, 1983, the relevant portions of which are incorporated herein by reference. See also Szoka, Jr. et al., (Ann. Rev. Biophys. Bioeng., 1980, 9:467), the relevant portions of which are also incorporated herein by reference.

The prolonged release of antibiotics is dependent, among other things, on the properties and sizes of the nanoparticles. A combination of various sizes of micelles, inverse micelles and liposomes (collectively, “nanoparticles’) is used herein to achieve the goal of prolonged release in view of the different half-life of each antibiotic. By manipulating the concentrations and sizes of the nanoparticles, controlled release of encapsulated antibiotics over time is achieved. The combination of inverse micelles and liposomes can be used, for example, for the encapsulation of any hydrophilic (water soluble) antibiotic such as vancomycin, gentamicin, gentamicin sulfate, tobramycin, ampicillin, penicillin, ethambutol, clindamycin, and a cephalosporin including cefazolin, ceftriaxone and cefotaxime for bacterial infections, acyclovir for viral infections, and amphotericin B for fungal infections. A combination of regular micelles and lipsosomes can be used for the encapsulation of hydrophobic antibiotics such as rifampicin, chloramphenicol, novobiocin, spectinomycin, trimethoprim (often supplied as a sulfamethoxazole), erythromycin, doxycycline and minocycline.

The present invention relates to a nanosystem capable of releasing drugs in a controlled manner using a combination of unilamellar and multilamellar liposomes along with regular and inverse micelles containing antibiotics. The alternating release times of these nanoparticles allow sustained antibiotic delivery over a specified time period. Liposomes and micelles are a completely biodegradable and non-toxic drug delivery system that has been extensively studied since 2000 for the ability to deliver therapeutic drugs. (Arkadiusz et al., 2000).

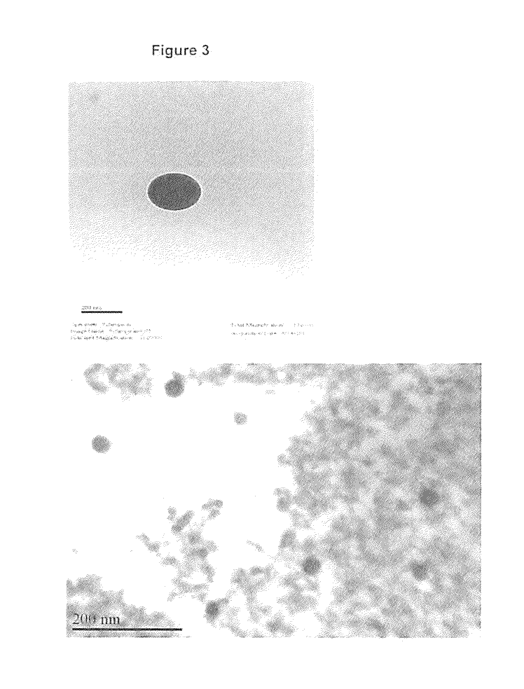

Unilamellar and multilamellar liposome vesicles, according to the present invention, are prepared using modified published methods such as reverse-phase evaporation and lipid hydration technique. (Mugabe et al., 2006a, Mugabe et al., 2006b, Otilia et al, 2005., Rawat et al, 2006). Referring to FIG. 3, rifampicin, a hydrophobic drug, is effectively encapsulated inside the nanoparticles. Various molar ratios of rifampicin and o-(decylphosphoryl)choline are first dissolved in methanol. The methanol is removed by rotary evaporation (45° C., 150 revolutions/min and 600 mm of Hg vacuum under a stream of Argon) to form a dry film. The film is rehydrated by vortexing for about 5 min and sonicating for about 5 min with about 0.01 mol/L acetate buffer (pH 5). The resulting aqueous dispersion is equilibrated in the dark for about 2 hours at about 25° C., and the excess drug is removed by centrifugation before characterization.

Double emulsion solvent extraction technique is also used to create drug delivery vehicles. PLGA (polylactic-co-glycolic acid) and 5% (w/v) polyethyleneglycol (PEG) is dissolved in about 2 ml of dichloromethane (DCM) separately. Suitable polymers generally include polyethyleneglycol, polylactic and polyglycolic acids, and polylactic-polyglycolic and copolymers having a molecular weight between about 1,000-5,000 daltons. About 3 ml of rifampicin stock solution in PBS is measured using a drug to polymer ratio of 1:20. Both the drug and the polymer solutions are mixed with a high speed vortex mixer to form a stable emulsion. About 100 ml of 0.2% (w/v) aqueous polyvinylchloride solution is prepared by continuous stirring in moderate heat for about 1 hr. Afterwards the drug-polymer emulsion is poured into polyvinyl alcohol (PVA) solution which leads to the double emulsification of the particles. The mixture is sonicated for about 30 minutes and the particles are collected by centrifugation for about 15 minutes at about 13,000 rpm. The particles are washed with deionized water twice after the supernatant is discarded and are then resuspended in water and stored under refrigeration before Transmission Electron Microscopy (TEM) imaging as shown in FIG. 3. The upper scan shows encapsulated rifampicin nanoparticles. The lower scan shows no aggregation of the nanoparticles within the matrix.

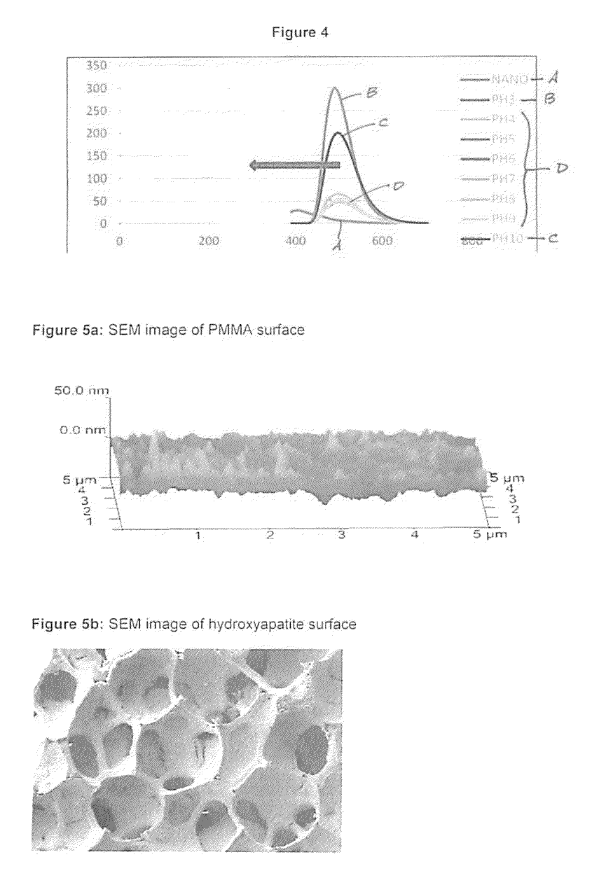

To ensure the drug rifampicin is indeed encapsulated within the vesicles, fluorescent spectroscopic analysis is conducted. FIG. 4 shows the fluorescent spectrum before (B-D) and after (A) the encapsulation. Not only did the fluorescent intensity dramatically decrease at the same concentration after the encapsulation, rifampicin nanoparticles also showed a blue shift (decreased wavelength) in the spectra which indicated the solvent environment had shifted from a hydrophobic environment to a more hydrophilic, polar environment. This data further supports encapsulation.

The chemical composition, total molecular weight and head/tail length ratios of micelle and liposomal monomers can be changed and modified in order to optimize the size, characterization and morphology. Moreover, this nanosystem can be customized according to the needs of the patient by varying entrapped antibiotics and the mixture of nanostructures. Finally, all nanovesicles in this system comprise organic materials, which are already used in many FDA approved drug delivery systems.

In order to provide antibiotic drug transport directly to the surgical site and, attain optimal nanoparticle stability, a polymer coating that contains antibiotic-encapsulated nanoparticles is applied over, for example, a PMMA implant or a bone grafting material. Common implant materials such as PMMA and hydroxyapatite have a very high surface area (FIGS. 5a and 5b) which provides a substantial amount of attachment area for the nanoparticles. First, a nanoparticulated drug-cocktail is mixed with a polymeric coating material and is then dissolved in a carrier solvent (commonly water or an alcohol). A thin film of nanoparticle-containing polymer is then brushed on the upper surface of the implant material which will set quickly using conventional UV light or chemical curing methods. When the carrier evaporates, the antibiotic-containing nanoparticles are stably attached to the surface providing sustained, localized release of the drug. Polymers suitable for use as coating materials according to the present invention include water-based polyvinylpyrrolidone, alcohol-based polymethylacrylate isobutene mono-isopropylmaleate, and hexamethyldisiloxane or isooctane solvent-based siloxane polymers.

Thus, as described herein, the present invention relates to a pharmaceutical formulation comprising nanoparticles containing a therapeutically effective amount of at least one antibiotic and a physiologically acceptable coating material whereby application of the formulation to an implant before surgery provides for extended release of the antibiotic to treat infection.

As used herein, a “therapeutically effective amount” of the antibiotic is an amount sufficient to provide the equivalent effect in a human of oral administration of the antibiotic in a range between about 1 mg/kg body weight and about 15 mg/kg body weight, more preferably between about 2 mg/kg body weight and about 10 mg/kg body weight. For example, the amount of antibiotic in a PMMA cement is usually several grams of antibiotic per 40-50 grams of PMMA powder depending on the total surface area of the implant and the particular antibiotic used. The amount of antibiotic used in the nanoparticles of the present application is substantially less than that per unit area.

In a preferred embodiment, the antibiotic is selected from the group consisting of rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof. The implant is formed of a material preferably selected from the group consisting of polymethylmethacrylate, hydroxyapatite and copolymers thereof. The implant can comprise a cranial implant formed of polymethylmethacrylate or a cranial bone graft formed of hydroxyapatite.

Also, in a preferred embodiment, the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate. More preferably, the physiologically acceptable coating material comprises polyvinylpyrrolidone as a first component admixed with nitrocellulose as a second component.

A method for the release of antibiotics from the implant over an extended period of time comprises providing an above-identified antibiotic-containing nanoparticle formulation and applying the formulation to the implant before surgery.

In another embodiment, a pharmaceutical formulation comprises first nanoparticles containing a therapeutically effective amount of a first antibiotic; second nanoparticles containing a therapeutically effective amount of a second antibiotic; and a physiologically acceptable coating material. Application of the formulation to an implant before surgery provides for extended release of the first and second antibiotics to treat infection.

The first antibiotic can be hydrophobic and is selected from the group consisting of rifampicin, chloramphenicol, novobiocin, spectinomycin, trimethoprim, erythromycin, doxycycline and minocycline, including pharmacologically acceptable salts and acids thereof. The second antibiotic can be hydrophilic and is selected from the group consisting of vancomycin, acyclovir, amphotericin B, gentamicin, gentamicin sulfate, tobramycin, ampicillin, penicillin, ethambutol, clindamycin and cephalosporins including cefazolin, ceftriaxone and cefotaxime, including pharmacologically acceptable salts and acids thereof. In the alternative, both antibiotics can be hydrophobic or both antibiotics can be hydrophilic.

The corresponding method provides for the extended release of antibiotics from the implant comprising providing an above-identified first and second antibiotic-containing nanoparticle formulation and applying the formulation to the implant before surgery.

This specific targeting drug delivery system can help reduce dangerous side effects. It also eliminates the time that is otherwise needed for the drugs to be processed by the liver. Therefore, a lesser amount of drug will have the same beneficiary effects compared with the drugs being administered intravenously or orally. Clindamycin has been a primary antibiotic used in blast-injured patients, as it is effective against both aerobic and anaerobic bacterial infections. Usually clindamycin is administrated orally, absorbed through the gastrointestinal tract, extensively metabolized in the liver, and then distributed throughout the body. Only a small therapeutic concentration (between 5 and 10 percent) can be achieved in the brain after 1.5 to 5 hours after administration of the drug. Since it has to be systematically circulated, a much higher initial dose is required for the effective dosage to reach the brain. A higher initial dosage leads to more severe side effects such as headache, bloody diarrhea, fever, nausea, severe blistering of the skin and jaundice which all can be reduced to a minimum by administrating the effective dosage directly to the infected area according to the present invention.

### Materials and Methods

### Cranial Implants Formed of Polymethylmethacrylate (PMMA) Formulations

The human skull includes two major parts, the cranium and the facial skeleton. The cranium, which carries and protects the brain, comprises eight bones: the occipital, two parietals, the frontal, two temporals, the sphenoid and the ethmoid. In cranial implantation, the parietal bones are the most commonly replaced by artificial materials.

The following formulations are examples of PMMA copolymers suitable for use as materials in forming cranial implants according to the present invention.

TABLE 1

| | PMMA | MMA | EMA | IMA | MEKP |
|---|---|---|---|---|---|
| Formulation | (grams) | (ml) | (ml) | (ml) | (ml) |
| A | 18 | 12 | — | — | — |
| B | 18 | 12 | 8 | — | — |
| C | 16 | 12 | — | 8 | 2 |
| D | 20 | 15 | — | 14 | 0.75 |

PMMA polymethylmethacrylate
MMA methyl methacrylate
EMA ethyl methacrylate
IMA isobutyl methacrylate (Acryloid B-67, an adhesive)
MEKP methyl ethyl ketone peroxide, a catalyst

The components of each formulation are thoroughly mixed separately on a hotplate in a chemical hood with constant stirring. The mixture is cast in an aluminum or tin molding plate with the desired thickness. The typical adult skull is about 5.0 to 8.0 mm thick—a female skull is usually about 7.1 mm thick and a male skull is usually about 6.5 mm thick. A pediatric skull, on the other hand, is about 2.0 mm thick. The molding plate is placed in an oven (about 2 hours for an adult implant and about 1 hour for a pediatric implant) at about 80° C. to cure the PMMA. The tensile strength of PMMA implants is tested using an Instron machine. Tensile strength is generally measured in N/cm². The normal human skull has a tensile strength of 7,053 N/cm². The tensile strength of each of Formulations A-D is either equal to or greater than the average tensile strength of the human skull.

### EXAMPLES 1A AND 1B

### Unilamellar Liposome Formulation (Water-Oil-Water (w/o/w) Emulsion)

Using the phase-transfer method, the organic phase (vitamin F or vitamin E) is loaded with L-α-phosphatidylcholine or palmitic acid (surfactants). Palmitic acid has a critical micelle concentration (CMC) of about 8.0 g/L. Surfactants that have low CMC values are more suitable for emulsion formations because they can be used in smaller amounts relative to other surfactants with higher CMC values, and produce the same desired effect. Therefore, a surfactant such as stearic acid (3.8), oleic acid (5.0) and linoleic acid (2.5) can also be used in this formulation. The hydrophilic drug vancomycin (anti-bacterial) or acyclovir (anti-fungal) is dissolved in water. The water phase is titrated dropwise into the organic phase with constant stirring under low heat. This procedure creates a water-in-oil (w/o) emulsion, and reverse micelles are formed within the emulsion. With the aqueous drug solution encapsulated inside the micellar core, this w/o phase is again titrated drop by drop with a final aqueous phase containing its particular surfactant (L-α-phosphatidylcholine or palmitic acid). The final product is a water-oil-water emulsion. Phase separation occurs only when the concentration of any phase has exceeded the equilibrium. Liposomes can be created by sonication. Low shear rates create multilamellar liposomes, which have multiple layers, like an onion. Continued high-shear sonication tends to form smaller unilamellar lipsomes.

A water-in-oil (w/o) emulsion is prepared as the primary oil phase. As Example 1A, 5 ml (4.94 g) of α-tocopherol is mixed with 0.0019 g of L-α phosphatidylcholine (500 μM). As Example 1B, 5 ml of α-tocopherol (4.72 g) is mixed with 0.006 g of palmitic acid (500 μM). Dissolve either a luminescence marker or a hydrophilic drug in water to form the aqueous phase. The aqueous phase is titrated into the primary oil phase with constant stirring under low heat. The mixture is then sonicated for about one hour, and then centrifuged for about 15 minutes at 13,000 rpm to form a water-in-oil (w/o) emulsion. The fluid is discarded.

A final aqueous solution of surfactant L-α phosphatidylcholine or palmitic acid, respectively, is prepared. In Example 1A, 22 ml of de-ionized water is mixed with 0.0043 g of L-α phosphatidylcholine (250 μM). In Example 1B, 22 ml of de-ionized water is mixed with 0.0014 g of palmitic acid (250 μM).

Each of the above water-in-oil emulsions is titrated into the above corresponding final aqueous solution with constant stirring. Each resulting mixture is sonicated for about one hour under high sheer to create the respective unilamellar water-oil-water liposome.

### EXAMPLE 2

### Multilayer Liposome Formulation (Water-Oil-Water-Oil (w/o/w/o) Emulsion)

An organic phase is prepared by dissolving 500 mg of AOT (sodium 1,4-bis[(2-ethylhexyl)oxy]-1,4-dioxobutane-2-sulfonate) in 4 ml of ethyl acetate. A hydrophilic drug (23 μM of fluorescein dye used as an indicator) is dissolved in about 1 ml of water to form an aqueous phase. The aqueous phase is titrated dropwise into the organic phase with constant stirring. Reverse micelles are formed within this water-in-oil (w/o) emulsion. After mixing, 2 ml of the organic phase is evaporated, resulting in a water-in-oil emulsion having a total volume of 3 ml.

The final water phase is formed by dissolving 500 mg of AOT in 40 ml of water. AOT is only slightly soluble in water. If desired, a more hydrophilic polymer such as phosphocholine and palmitic acids can be used in this step. The above water-in-oil emulsion is added dropwise into the final water phase to form a water-oil-water (w/o/w) liposome.

Because the coating materials require an organic solvent as a carrier, the foregoing liposome is suspended in the organic phase in order to be homogenously mixed with the coating polymer. The final organic phase is formed by dissolving 500 mg of AOT in 10 ml of ethyl acetate.

The foregoing water-oil-water liposome is added to the final organic phase to produce multi-layer w/o/w/o liposomes.

### EXAMPLE 3

### Reverse Micelles (Water-oil (w/o) Emulsion)

2.2 grams of AOT is mixed with 5 ml of vitamin E (or vitamin F) with gentle heating and continuous stirring. Once the AOT is dissolved, a water phase comprising 2 ml of water and the drug is added dropwise with constant stirring. The mixture is then sonicated for 15 minutes.

Examples 1A, 1B, 2 and 3 are designed to encapsulate and deliver hydrophilic drugs. Hydrophobic drugs are known to be more difficult to transport into targeted cells. The present invention also provides a unique system for encapsulating and delivering hydrophobic antibiotics.

### EXAMPLE 4

### Nanoparticles (Oil-Water-Oil (o/w/o) Particles)

A first oil phase is formed by dissolving the hydrophobic anti-bacterial drug chloramphenicol in 5 ml of ethyl acetate. The first oil phase is then titrated into a 5 ml water phase containing 500 μM palmitic acid with constant stirring. Micelles are formed in this o/w emulsion.

Stirring is continued until all of the ethyl acetate from the first oil phase is evaporated by bringing the volume down to about 5 ml which conforms to the original volume of the water phase. Nanoparticles are formed with chloramphenicol entrapped inside the micelles.

The final oil phase is formed by dissolving 12 or 18% w/v of polycaprolactone (PCL) and 500 μM palmitic acid in ethyl acetate with stirring at 50° C. The final oil phase is removed from the heat and the above nanoparticles are added dropwise into the final oil phase with constant stirring. Stirring is continued until the solution reaches room temperature. PCL fibers then form a thin layer surrounding the double layered nanoparticles.

### Coating Material

About 0.7 grams of polyvinylpyrrolidone (PVP) is dissolved in about 0.7 ml of ethanol to form a mixture. About 0.7 ml of the resulting mixture is mixed with about 9.3 ml of nitrocellulose. A liquid bandage usually includes about 7% alcohol in its formulation. The present coating formula follows the common liquid bandage formulation with slight modifications. FDA-approved materials are used in order to avoid the long governmental approval and evaluation process. Thus, the final product is readily available for use by consumers. Nitrocellulose is used as the primary ingredient in the coating material. When nitrocellulose is dissolved in ether or alcohol, a collodion is formed. When the collodion dries, a flexible cellulose film is produced. Besides nitrocellulose, 2-octyl cyanoacrylate and n-butyl cyanoacrylate can also be used as the primary ingredient in the coating formulation. The main advantage of these materials is they do not break down in the body to form toxic byproducts.

### Stabilizer and Thickening Agent

2.5% w/v of human collagen can be added to each of the above examples as a stabilizer. Carbomer, a synthetic high molecular weight polymer of acrylic acid, is used as a thickening agent to increase the viscosity of the formulations.

### Binding Agents

Different binding agents can be used in the present coating formulations. The binding agents (binders) can secure nanoparticles and develop adhesion to the implant surface. The present coating methodology involves a crosslinking film formation—the highest-performance coating films are based on reacting polymer precursors to build up a three-dimensionally crosslinked network. At least the following types of natural binders can be added to the nitrocellulose matrix:

Drying oils: Natural products such as linseed (flax seed) oil, tung oil or boiled linseed oil contain at least 50% unsaturated fatty acid triglycerides. When reacted with oxygen in the air, these oils crosslink to form network polymers. Adding oxygen to fatty acids and the subsequent formation of hydroperoxide derivatives of the fatty acids is a very complicated process that happens naturally when the oils are exposed to atmospheric oxygen. Oxidation hardens the drying oil at room temperature. Adding 10 to 30% v/v of boiled linseed to the present coating formulations enhances the adhesion of the coating material to the implant surface and provides even coating.

Adhesion promoters: High molecular weight polyethylene glycol 3000 or a natural resin such as gum rosin and rosin ester can be added to the coating material to strengthen its adhesive properties. Rosin is a treated resin from which one of its constituents, terpene has been removed. Rosin is very compatible with drying oil, therefore both can be used together in the formulation. The darker the rosin, the softer it is. There are many different derivatives of rosin and rosin ester; polymerized rosin is preferred herein for improving the adhesive ability of the coating.

### Results

### PMMA Implant

SEM (Scanning Electron Microscope) images of the surfaces of PMMA implant materials described in Table 1 show different depth of grooves which are capable of embedding the liposomes and nanoparticles.

### Liposomes and Nanoparticles

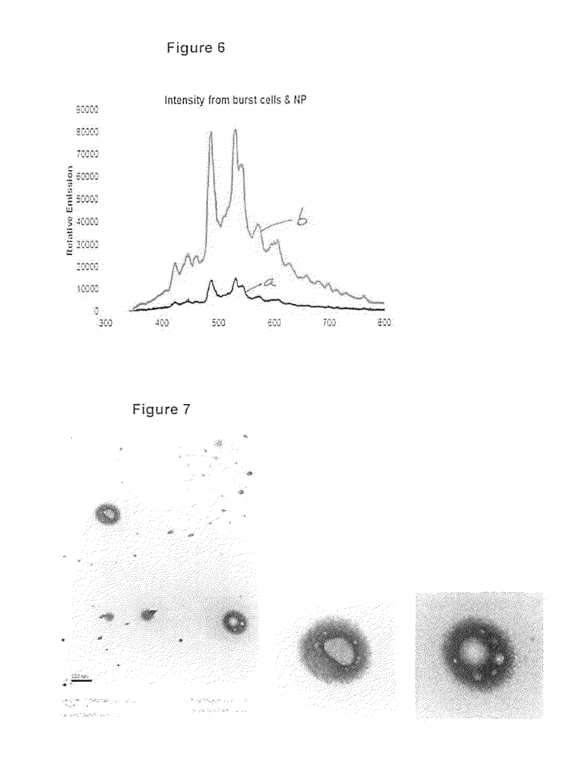

FIG. 6 shows the results of a cell penetrating study using human dermal fibroblast cells. After 2 hours incubation with the nanoparticles, the cell membranes are lysed using a 5 percent N-lauryl sacrosine sodium salt solution. Fluorescent readings were compared before (a) and after (b) cell lysis demonstrating the increased fluorescent intensity from burst cells and the cell uptake of the fluorescent marker.

In particular, this illustrates that the present nanoparticles have evidently diffused into the cells and released the cell contents into the cytoplasm. The hydrophobic antibiotic rifampicin has been successfully encapsulated inside the nanoparticles (FIG. 4). These rifampicin nanoparticles are then incubated [12 mg/ml of nanoparticles suspended in 3 ml of aqueous phosphate buffered saline (PBS)] with human cell culture (in 60 mm petri dish˜30,000 cells) for two hours at room temperature. After incubation, the fluid is discarded and the cells are washed with sterile aqueous PBS twice. After all the excess fluid is discarded, 3 ml of lysis solution (5.0% sodium N-lauroylsarcosine) is added to the cell culture in order to break the cell membrane. Emission spectra are taken before the incubation period and after the cell membranes are lysed. Line a in FIG. 6 shows all the drugs are encapsulated inside the nanoparticles. Line b shows the nanoparticles have diffused into the cells and rifampicin has been released from the nanoparticles into the cellular cytoplasm. The increasing emission intensity in line b compared to the original (before incubation) emission in line a demonstrates that the rifampicin is no longer being encapsulated inside the nanoparticles.

FIG. 7 shows TEM images of unilamellar liposomes formed according to Example 1B. At a magnification of 150,000×, one can clearly see the different layers of w-o-w liposome. Each liposome is about 100 nm in diameter and the arrows indicate the different components of the liposome. Arrow a identifies the interior of the liposome where the drugs are actually encapsulated. Arrow b indicates the first layer of the liposome. Arrow c shows the center space of the liposome which is filled with oil droplets. Arrow d indicates the outer layer of the liposome. FIG. 7 is the real microscopic image of FIG. 1.

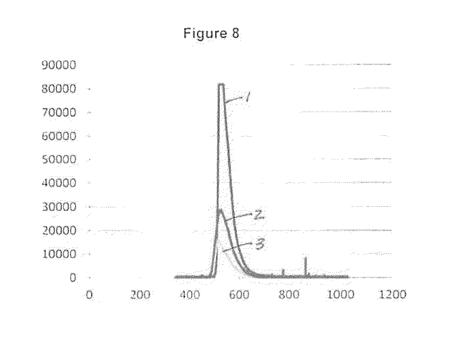

FIG. 8 shows that fluorescein dye in aqueous solution provides an intense peak (1).

The same concentration of fluorescein dye solution encapsulated within a reverse micelle formed according to Example 3 provides a less intense peak (2). The same concentration of fluorescein dye solution encapsulated within a liposome formed according to Example 2 provides an even less intense peak (3) demonstrating the relative uptake of the dye by nanoparticles according to the present invention.

Thus, FIG. 8 shows that at the same concentration, fluorescent intensities of fluorescein dye are very different in an aqueous solution, inside the reverse micelles or inside of liposomes. When the free fluorescein molecules are dissolved in an aqueous solution, the fluorescent intensity provides an intensive peak (line 1). When the same concentration of fluorescein dye is encapsulated inside the reverse micelles of Example 3, the intensity decreases (line 2). Due to the multilayer nature of liposomes, the same concentration of fluorescein dye solution trapped inside the liposomes of Example 2 provides the least fluorescent intensity (line 3).

Electron microscopy (EM) is commonly used to capture high-resolution imaging of liposomes and nanoparticles. However, EM requires that samples be placed in a vacuum and is not suitable for examining wet samples. The only means of imaging a wet sample with EM is to freeze or dry it, thus changing its nature in the process. According to the present invention, the QuantomiX capsules methodology developed by WETSEM® is used. (Electron Microscopy Sciences, Hatfield, Pa.). This technique eliminates many of the artifacts that result when preparing wet samples for EM. This technology also enables imaging of the present samples that contain oily and volatile solvent.

### Controlled Release Study

Human cerebrospinal fluid (CSF) is used to examine the controlled release kinetics. A luminescence marker is encapsulated inside the present liposomes and nanoparticles instead of the antibiotic in order to monitor the controlled release. Riboflavin is used to simulate the hydrophobic drug, and fluorescein dye is used to simulate the hydrophilic drug. The final product (a PMMA implant coated with encapsulated luminescence marker) is submerged inside the human cerebrospinal fluid, and the fluorescence is measured at 0, 4, 8, 16, 24, 48 and 72 hours. The intensity of fluorescence indicates the amount of drug that is released from the coating polymer into the cerebrospinal fluid. For the hydrophilic drug, Example 1A and 1B release the drug first and Example 3 holds onto the drug longer for delayed release. For the hydrophobic drug, direct incorporation of the drug into the coating polymer is used for immediate release, and Example 4 is for the prolonged release due to slow biodegrading period of the PCL polymer that surrounds the nanoparticles.

### Cytotoxicity Test

The toxicity of the present formulations is examined using the standard BCA test. Human brain glial cells (SVG p12 cell line) are obtained from American Type Cell Collection (ATCC). The subculture procedures follow the protocol published by the ATCC. Each formulation is evaluated to ensure its safety.

### REFERENCES

Arkadiusz K, Gubernator J, Przeworska E, and Stasiuk M. “Liposomal drug delivery, a novel approach: PLARosomes.” Acta Biochim. Polo., 2000, 47, 639-649.
Garay-Jimenez J C; Gergeres D; Young A; Lim D V; Turos E. “Physical properties and biological activity of poly(butyl acrylate-styrene) nanoparticle emulsions prepared with conventional and polymerizable surfactants.” Nanomedicine: Nanotechnology, Biology, and Medicine (Nanomedicine), 2009: 5(4): 443-51.
Henry C M. “Cover Story; Drug Delivery” C&E News Washington, 2002, 80, 39-47.
Johanson C E, Duncan J A, Stopa E G, Baird A. “Enhanced Prospects for Drug Delivery and Brain Targeting by the Choroid Plexus-CSF Route.” Pharm. Res., 2005 July, 22(7):1011-37.
Mugabe C, Halwani M, Azghani A O, Lafrenie R M, Omri A. “Mechanism of Enhanced Activity of Liposome-Entrapped Aminoglycosides against Resistant Strains of Pseudomonas aeruginosa.” Antimicrobial Agents and Chemother., 2006, 50, 2016-2022.
Mugabe C, Azghani A O, Omri A. “Preparation and Characterization of Dehydration-rehydration vesicles loaded with aminoglycosides and microlide antibiotics.” Int. J Pharm., 2006, 307, 244-250.
Müller-Goymann C C. “Physiochemical characterization of colloidal drug delivery system such as reverse micelles, vesicles, liquid crystals and nanoparticles for topical administration.” European Journal of Pharmaceutics and Biopharmaceutics, 2004, 58, 343-356.
Nandi I, Bari M, Joshi H. “Study of Isopropyl Myristate Microemulsion Systems Containing Cyclodextrins to Improve the Solubility of 2 Model Hydrophobic Drugs.” 2003, 4, 1-9.
Koo O M, Rubinstein I, Onyuksel H. “Camptothecin in sterically stabilized phospholipid micelles: A novel nanomedicine.” Nanomedicine: Nanotechnology, Biology and Medicine, 1 (2005) 77-84.
Rawat M., Singh D., Saraf S. “Nanocarriers: Promising Vehicle for Bioactive Drugs.” Biol. Pharm. Bull., 29(9) (2006) 1790-1798.
Richter A, Olbrich C, Krause M, Hoffmann J, Kissel T. “Polymeric Micelles for parenteral delivery of Sagopilone: Physicochemical characterization, novel formulation approaches and their toxicity assessment in vitro as well as in vivo.” European Journal of Pharmaceutics and Biopharmaceutics, 2010: 75, 80-9.
Torchilin V P. “Structure and design of polymeric surfactant-based drug delivery system.” Journal of Control Release, 2001, 73, 137-172.
Wang C H, Wang W T, Hsiue G H. “Development of polyion complex micelles for encapsulating and delivering amphotericin B.” Biomaterials, 2009: 30(19): 3352-8.

Although the present invention has been disclosed with respect to particular nanoparticles, antibiotics, formulations and methods, it will be apparent that a variety of modifications and changes can be made without departing from the scope and spirit of the invention, as described and claimed herein.

What is claimed is:

1. A pharmaceutical formulation comprising nanoparticles containing a therapeutically effective amount of at least one antibiotic and a physiologically acceptable coating material whereby application of the formulation to an implant before surgery provides for extended release of the antibiotic to treat infection.

2. The formulation according to claim 1 wherein the antibiotic is selected from the group consisting of rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

3. The formulation according to claim 1 wherein the implant is formed of a material selected from the group consisting of polymethylmethacrylate, hydroxyapatite and copolymers thereof.

4. The formulation according to claim 1 wherein the implant comprises a cranial implant formed of polymethylmethacrylate.

5. The formulation according to claim 1 wherein implant comprises a cranial bone graft formed of hydroxyapatite.

6. The formulation according to claim 1 wherein the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate.

7. The formulation according to claim 1 wherein the physiologically acceptable coating material comprises polyvinylpyrrolidone as a first component admixed with nitrocellulose as a second component.

8. A method for providing extended release of antibiotics from an implant comprising:
a) providing a pharmaceutical formulation comprising nanoparticles containing a therapeutically effective amount of at least one antibiotic in a physiologically acceptable coating material; and
b) applying the formulation to the implant before surgery
whereby the antibiotic is released over an extended period of time to treat infection.

9. The method according to claim 8 wherein the antibiotic is selected from the group consisting of rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

10. The method according to claim 8 wherein the implant is formed of a material selected from the group consisting of polymethylmethacrylate, hydroxyapatite and copolymers thereof.

11. The method according to claim 8 wherein the implant comprises a cranial implant formed of polymethylmethacrylate.

12. The method according to claim 8 wherein the implant comprises a bone graft formed of hydroxyapatite.

13. The method according to claim 8 wherein the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate.

14. The method according to claim 8 wherein the physiologically acceptable coating material comprises polyvinylpyrrolidone as a first component admixed with nitrocellulose as a second component.

15. A pharmaceutical formulation comprising first nanoparticles containing a therapeutically effective amount of a first antibiotic; second nanoparticles containing a therapeutically effective amount of a second antibiotic; and a physiologically acceptable coating material whereby application of the formulation to an implant before surgery provides for extended release of the first and second antibiotics to treat infection.

16. The formulation according to claim 15 wherein the first antibiotic is hydrophobic and the second antibiotic is hydrophilic.

17. The formulation according to claim 15 wherein the first antibiotic is selected from the group consisting of rifampicin, chloramphenicol, novobiocin, spectinomycin, trimethoprim, erythromycin, doxycycline and minocycline, including pharmacologically acceptable salts and acids thereof.

18. The formulation according to claim 15 wherein the second antibiotic is selected from the group consisting of vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

19. The formulation according to claim 15 wherein the implant is formed of a material selected from the group consisting of polymethylmethacrylate, hydroxyapatite and copolymers thereof.

20. The formulation according to claim 15 wherein the implant comprises a cranial implant formed of polymethylmethacrylate.

21. The formulation according to claim 15 wherein the implant comprises a cranial bone graft formed of hydroxyapatite.

22. The formulation according to claim 15 wherein the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate.

23. The formulation according to claim 15 wherein the physiologically acceptable coating material comprises polyvinylpyrrolidone as a first component admixed with nitrocellulose as a second component.

24. A method for providing extended release of antibiotics from an implant comprising:
a) providing a pharmaceutical formulation comprising first nanoparticles containing a therapeutically effective amount of a first antibiotic, second nanoparticles containing a therapeutically effective amount of a second antibiotic, and a physiologically acceptable coating material; and
b) applying the formulation to the implant before surgery
whereby the antibiotics are released over an extended period of time to treat infection.

25. The method according to claim 24 wherein the first and second antibiotics are selected from the group consisting of rifampicin, chloramphenicol, novobiocin, spectinomycin, trimethoprim, erythromycin, doxycycline, minocycline, vancomycin, acyclovir, amphotericin B, gentamicin, gentamicin sulfate, tobramycin, ampicillin, penicillin, ethambutol, clindamycin, and cephalosporins including cefazolin, ceftriaxone and cefotaxime, including pharmacologically acceptable salts and acids thereof.

26. The method according to claim 24 wherein the implant is formed of a material selected from the group consisting of polymethylmethacrylate, hydroxyapatite and copolymers thereof.

27. The method according to claim 24 wherein the implant comprises a cranial implant formed of polymethylmethacrylate.

28. The method according to claim 24 wherein the implant comprises a cranial bone graft formed of hydroxyapatite.

29. The method according to claim 24 wherein the physiologically acceptable coating material comprises a first component selected from the group consisting of polyvinylpyrrolidone, polymethylmethacrylate isobutene mono-isopropylmaleate, hexamethyldisiloxane and isooctane solvent-based siloxane polymers and copolymers thereof admixed with a second component selected from the group consisting of nitrocellulose, 2-octyl cyanoacrylate and n-butyl cyanoacrylate.

30. The method according to claim 24 wherein the physiologically acceptable coating material comprises polyvinylpyrrolidone as a first component admixed with nitrocellulose as a second component.
